(19) United States
(12) Patent Application Publication
(10) Pub. No.: US 2012/0180795 A1
(43) Pub. Date: Jul. 19, 2012

(54) Two Sided Pad

(76) Inventor: Steven W. Knight, Montgomery, TX (US)

(21) Appl. No.: 13/366,430

(22) Filed: Apr. 2, 2012

Related U.S. Application Data
(60) Provisional application No. 61/524,466, filed on Aug. 17, 2011.

(51) Int. Cl. A61M 16/06

(52) U.S. Cl. 128/206.24

(57) ABSTRACT

A cushioning pad includes two layers of different material. The pad is for use as a pad to be positioned between a mask and the face of a person when the mask is worn by the user. The materials for the two layers may include a gel material and a cloth material. This construction allows the user to select which layer will contact the user's face during use of the mask. The pad also serves to form a seal between the mask and the face of the user thereby improving the effectiveness of the mask.

This application claims priority to U.S. Provisional Application Ser. No. 61/524,466 filed on Aug. 17, 2011.

### BACKGROUND OF INVENTION

1. Field of the Invention

This invention relates to a cushioning pad adapted to be between a mask for example a mask used in conjunction with a continuous positive airway pressure (CPAP) machine and the face of a user. The pad may also be used in conjunction with any other type of mask that is designed to be placed on a person's face.

2. Description of Related Art

Cushioning pads for masks have been used that are formed of a foam or fabric material. For some patients gel or foam may be more comfortable whereas others may find fabric to be preferable. See for example publication number US 2011/0005524A1. The pad is configured for the nasal bridge region of the user or it may be configured to fit around the nose and mouth of the user.

### BRIEF SUMMARY OF THE INVENTION

The invention disclosed and claimed in this application is for a cushioning pad adapted to be placed between a mask and the face of a user. The pad is constructed so that one side of the pad is formed from a first material and the other side of the pad is formed of a different material. Thus a single pad is provided that allows the user to choose the material for contact with the skin that is most comfortable for the user. The type of material may include cloth, gel, foam, fabric, lambs wool, or any other type of material that is comfortable to a user's skin.

### BRIEF DESCRIPTION OF THE SEVERAL VIEWS OF THE DRAWING(S)

FIG. 1 is a perspective view of an exemplary embodiment of the present invention;

FIG. 2 is a front view of the exemplary embodiment of the present invention in use with a first side up;

FIG. 3 is a front view of a second embodiment of the present invention in use with a second side up;

FIG. 4 is a front view of an embodiment of the invention.

FIG. 5 is a front view of the exemplary embodiment of the present invention with a first side up and a second side down;

FIG. 6 is a cross sectional view taken along line 6-6 of FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

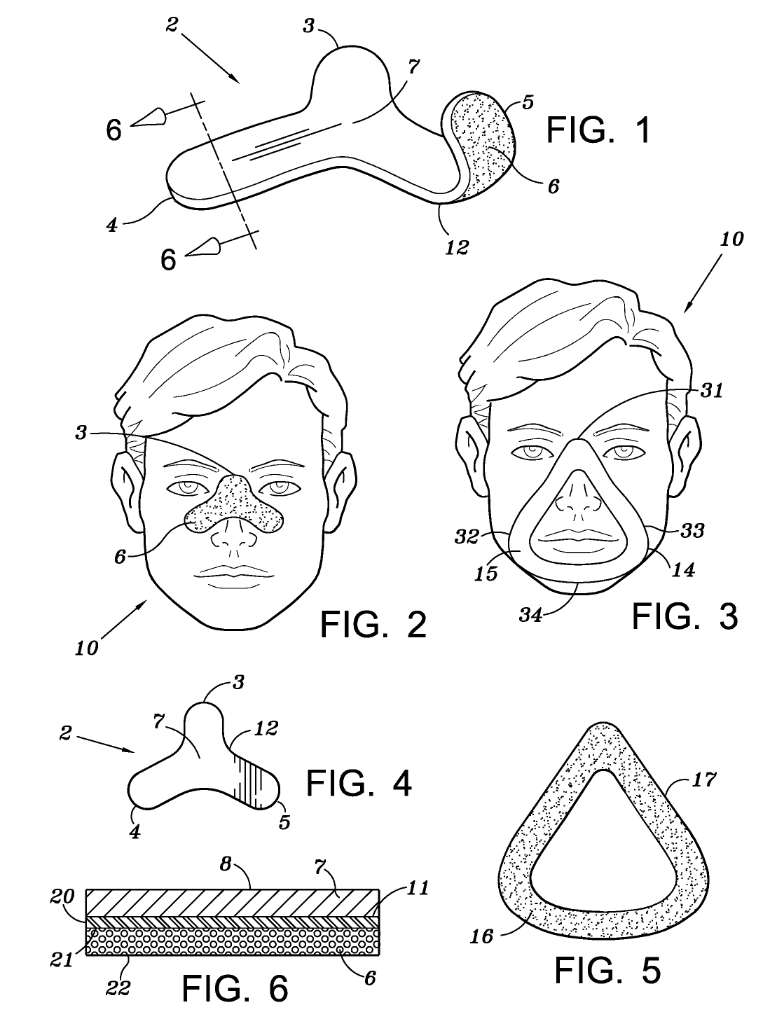

Broadly, an exemplary embodiment of the present invention generally provides a pad as shown at 2 in FIG. 1. More specifically, an exemplary embodiment of the present invention provides a two sided pad that protects skin and seals a continuous positive airway pressure (CPAP) mask at the bridge of the nose.

As shown in FIGS. 1-6, a pad may comprise a cloth layer 7 and a gel layer 6. The cloth layer may have a first perimeter 12, a first top surface 8, and a bottom surface 11. The gel layer 6 may have a second top surface 21 and a second perimeter which borders the first perimeter of the cloth layer and may be adhered to at least the first perimeter of the cloth layer and the bottom surface of the cloth layer 11 by an adhesive as shown at 20 in FIG. 6, The cloth layer and the gel layer may be flexible to fit a contour of a face. Gel layer 6 has a bottom surface 22 that may contact the face of the user. Alternately the gel layer may be placed on the fabric layer in a molten state so as to partially penetrate the cloth layer.

An exemplary embodiment of the present invention may come in various sizes, colors, shapes, and models to fit different sizes of nasal or flu face mask. The gel layer and the cloth layer may be bonded together at 20 by heat, pressure, or glue, for example, giving the patient a choice of using it either cloth or gel side down, whichever is more comfortable.

The gel layer 6 and the cloth layer 7 may be the same size allowing the user to use it as a convertible, or to flip the unit to the side that makes the best seal or creates the most comfort. The gel and cloth may contain fragrance as a method of aromatherapy. The gel may contain moisturizers, lotions, fragrances, for example to promote healing to the skin around the nose and mouth.

An exemplary embodiment of the present invention may work by cushioning the skin around the face, including the bridge of the nose coming down below both eyes, under the eyes, the sides of the face, above the top lip, and below the bottom lip to prevent pressure sores and leaks from a positive pressure sleep apnea device as shown at 31, 32, 33, and 34 in FIG. 3.

FIG. 5 illustrates the bottom surface 16 of the gel layer according to the embodiment of FIG. 3.

The two-sided pad according to exemplary embodiments may be made by cutting or pressing out a certain shape of gel, and matching it with the same shape made from cloth. Flaps or thinner skins of cloth may be added to different positions to improve the sealing and sore prevention. Perforated areas may be added in case users want to include more than one size in a box. Perforated areas may be made by drawing or imprinting lines on the unit to show the customer where to cut it to achieve a different size with one unit.

The two-sided pad may be placed on the face at the same place where a CPAP mask fits to prevent leaks and pressure sores. As shown in FIG. 2, the pad may be shaped to fit over the upper portion of the nose and between the eyes. The pad includes two wing portions 4, 5, and a central portion 3. The pad may be used to make the seal better on medical masks that keep germs or harmful drugs or other substances out for a caregiver and also the patient. A suitable gel for use in this invention is a mineral oil based thermoplastic elastomer. Other suitable gels include polyurethanes and silicones, for example.

FIG. 6 illustrates an embodiment wherein the pad has a generally rectangular cross-section, however the side portions of each layer may be tapered so as to create a gradual transition from the face to the top of the pad.

An exemplary embodiment of the present invention may further be used as a seal in scuba masks, firefighter helmets, welder helmets, or goggles.

It should be understood, of course, that the foregoing relates to exemplary embodiments of the invention and that modifications may be made without departing from the spirit and scope of the invention as set forth in the following claims.

Although the present invention has been described with respect to specific details, it is not intended that such details should be regarded as limitations on the scope of the invention, except to the extent that they are included in the accompanying claims.

I claim:

1. A pad, comprising:
a first layer having a first top surface, and a first bottom surface;
a second layer having a second top surface and a second bottom surface, wherein the first layer and the second layer are attached to each other and are flexible to fit a contour of a. face, and
the first and second layer is formed of different material.

2. The pad according to claim 1 wherein the first layer is formed of cloth material and the second layer is a gel material.

3. The pad according to claim 1 having a shape including a central portion and two wing portions so as to fit over the top portion of the nose and between the eyes of a user.

4. The pad according to claim 1 having a shape to fit over the top portion of the nose and around the lower lip of a user, and having a central opening to accommodate the nose and mouth of a user.

5. The pad according to claim 1 wherein the first layer is formed from the group comprising cloth, gel, foam, fabric, lambs wool and the second layer is formed from another material in the same group.
